(19) United States
(12) Patent Application Publication
(10) Pub. No.: US 2005/0143810 A1
(43) Pub. Date: Jun. 30, 2005

(54) Cardiovascular implant, method and device for its production, and its provision for surgery

(76) Inventors: Martin Dauner, Esslingen (DE); Michael Doser, Filderstatt (DE); Reinhard Kottler, Beuren (DE); Heinrich Planck, Nuertingen-Zizishausen (DE); Helmut Gulbins, Blaustein-Herrlingen (DE); Bruno Meiser, Muenchen (DE); Bruno Reichart, Leutstetten (DE)

Correspondence Address: NATH & ASSOCIATES, PLLC;Sixth Floor, 1030 15th Street, N.W., Washington, DC, 20005, US

(21) Appl. No.: 10/970,831

(22) Filed: Oct. 22, 2004

(30) Foreign Application Priority Data
Oct. 24, 2003 (DE) 10350287.4

(51) Int. Cl. A61F 2/24

(52) U.S. Cl. 623/2.12; 623/901; 623/1.26

(57) ABSTRACT

A cardiovascular implant for use in surgery, in particular a heart valve with flaps, made substantially of biocompatible synthetic polymer material in the form of a three-dimensional structure, wherein the polymer material is substantially nonabsorbable under physiological conditions, and the implant, at least on part of its surface, is formed as a microporous nonwoven from microfibers of the polymer material, which permit colonization with cells.

The present invention relates to a cardiovascular implant, a method for its production, a device for its production, and its provision for surgery.

In surgery, implants are used for supporting and for completely or partially replacing diseased organs, or organs that have been damaged by trauma. Such implants or prostheses can be made using metals, plastics, biopolymers, or biological material from animals or humans.

Problems arise with implants because the body of the recipient patient recognizes the implant as a foreign body and activates defense mechanisms, which can lead to functional failure and rejection of the implant.

Implants made of metal, plastic or composite material are mechanically strong, but their surfaces promote the formation of blood clots, so that the patients have to take blood-thinning medicines (anticoagulants) on a permanent basis, which in turn can lead to bleeding complications (haemorrhage).

Implants obtained from biological material, for example from pigs or cattle or from human donors, do not present complications of thromboembolism. However, when implanting biological tissue not originating from the actual patient, immunological reactions may occur which lead to the implant degenerating within a period of 10 to 20 years after implantation, and renewed surgery then has to be performed on the patient, who is of course also older by then.

Attempts to replace blood vessels or heart valves by cell colonization of absorbable support structures fail because of the inadequate strength of the newly formed tissue.

In the case of implants for use in the cardiovascular system, for example vascular prostheses or heart valves, it is particularly important to ensure a reliable and long-lasting function without risk of further impairment to health.

It is therefore an object of the invention to make available an implant which overcomes the problems and disadvantages of the implants of the prior art, is able to function permanently and reliably when implanted in the patient, and can be produced easily and inexpensively by medical engineering techniques.

This object is achieved by a cardiovascular implant for use in surgery, in particular a heart valve with flaps, made substantially of biocompatible synthetic polymer material in the form of a three-dimensional structure, wherein the polymer material is substantially nonabsorbable under physiological conditions, and the implant, at least on part of its surface, is formed as a microporous nonwoven from microfibers of the polymer material, which permit colonization with cells. This ensures permanent support of the tissue, which is necessary, inter alia, in the high-pressure region (arterial region).

The polymer material according to the invention is advantageously characterized in that the porous structure of the implant is able to promote metabolic exchange and, in particular, adherence of cells. The pore size of the micropores can preferably lie in the range of from 0.1 to 20 μm, in particular 1 to 8 μm.

Advantageously, the polymer material can be at least in part one that is based on polyurethane (PUR). In particular, the polyurethane can be a linear polyurethane. The polyurethane can preferably contain silicone polyurethanes as chain extenders. Examples of polyurethanes to be used according to the invention are segmented aromatic and aliphatic polyurethanes which comprise hard segments selected from methylene bis-(4-cyclohexyl) isocyanate and 4,4′-methylene bisphenyl diisocyanate, soft segments selected from polytetramethylene ether glycol, polycarbonate, polytetramethylene oxide/polydimethylsiloxane and polycarbonate/polymethylsiloxane, and chain extenders selected from ethylenediol and 1,4-butanediol. The molecular weights can be from 50 kD to 300 kD. Shear viscosities for illustrative polymers according to the invention can be in the region of 66 mPas at a solution concentration of 5% and up to 1294 mPas at a solution concentration of 10%.

In another embodiment of the invention, the polymer material can be at least partly based on polyethylene terephthalate (PET). Further polymers that can be used according to the invention are polyurethane (PUR), thermoplastic polyurethane (TPU), thermoplastic elastomer (TPE) and polyether block amide (PEBA). The polymer material of the heart valve according to the invention is particularly advantageously distinguished by its high elasticity. Likewise, the polymer material of the heart valve according to the invention can be distinguished by its high flexibility.

In a particular embodiment of the invention, the polymer material can be partially absorbable under physiological conditions.

The structure, particularly in the area of the heart valve flaps, can advantageously be made of polymer fiber material as a textile nonwoven, in particular as a spray-bonded nonwoven. In another embodiment, the polymer fiber material as nonwoven can be produced by an electrostatic method of producing nonwovens. In yet another embodiment, the polymer fiber material as nonwoven can be produced by a meltblown technique. As an alternative embodiment, the polymer fiber material can be formed as a nonwoven using a combination of the aforementioned nonwoven production techniques.

The nonwoven structure can preferably have a surface roughness.

In the implant according to the invention, the textile material can have a thickness of 10 to 500 μm, in particular of 30 to 70 μm. The microfibers can preferably have a thickness of 0.1 to 10 μm. In addition, the microfibers can have a length of 5 to 25 mm. According to the invention, the nonwoven can preferably have a pore size in the range of the fiber thickness. Advantageously, the microfibers can have a length of 0.1 to 10 μm between connection points. The microfibers can preferably be connected to one another by adhesive bonding, in particular by welding.

In a development of the invention, an implant can be made available in the form of a heart valve prosthesis comprising a tubular portion as a stump of a cardiac blood vessel, in particular an aortic stump, which is designed to be sutured onto an end piece of a natural blood vessel, three flaps which form a heart valve being arranged on the stump and being movable and interacting with one another in the manner of a valve so as to open and close upon flow of blood. In this way, it is possible to make available a complete heart valve prosthesis with valve flaps, bulbi and sinus.

The heart valve flaps advantageously imitate a natural heart valve. The heart valve flaps can preferably have a thickness decreasing toward the center of the heart valve. The construction of the flaps and that of the portion forming the blood vessel are preferably chosen so as to promote vascularization. At least the flaps are preferably made completely of nonwoven material.

In such a heart valve prosthesis, the heart valve flaps can be made of a planar nonwoven. In particular, the heart valve flaps can be arched. Advantageously, the heart valve flaps can be shaped thermally and/or upon production. The heart valve flaps in the implant according to the invention can have a thickness of from 10 to 500 μm. In particular, the implant can have a wall thickness of from 1 to 3 mm in the area of the vessel portion.

In one embodiment of the invention, the heart valve flaps can be produced separately and secured on the tubular portion of the prosthesis, in particular adhesively bonded. The flaps can be bonded with liquid adhesive, in particular polyurethane adhesive. The flaps can preferably be bonded in with polymer from the nonwoven production. In another embodiment of the invention, the heart valve flaps can be produced separately and secured on the tubular portion of the prosthesis, in particular melted on to it. This securing by melting of the polymer material can be achieved by ultrasonic welding.

In another embodiment of the invention, the implant, in particular the heart valve prosthesis, especially the tubular portion, can be made in one piece with the flaps.

According to the invention, the implant, in particular the heart valve prosthesis, can be produced directly in a three-dimensional form. For this purpose, electrostatic techniques, shaping of preforms and tempering may be mentioned by way of example. In one embodiment, the implant can be produced at least partially by a spray technique. In another embodiment, the implant can be produced at least partially using a known electrostatic spinning technique. The implant can be produced using a combination of techniques of spraying and spinning of microfibers. In one development, the heart valve prosthesis according to the invention can be provided with a suture ring.

In an advantageous embodiment of the invention, different parts of the implant, in particular the heart valve flaps and the tubular portion, can be made in different textile constructions and/or of different materials. The heart valve flaps can preferably be made as a textile nonwoven-type membrane. In one embodiment, the tubular blood vessel portion can be made as a textile warp knitted fabric. In another embodiment, the tubular blood vessel portion can be made as a textile woven. It is preferable for the entire surface of the implant to be made as a nonwoven, in particular the entire implant itself.

In one advantageous embodiment, the heart valve flaps and the tubular portion can be made in different pore sizes. The different pore sizes can be of advantage in terms of cell colonization. In the case of the heart valve flaps, surface colonization is to be favored. In the case of the blood vessel portion, inward penetration of cells is to be favored.

In an alternative embodiment, the heart valve flaps and the tubular portion can be made of different materials. The materials can advantageously differ in terms of their physical and/or mechanical properties, for example in terms of flexibility and elasticity. In this way, it is possible to meet the requirements for optimal functioning of the heart valve prosthesis under physiological conditions in the patient's body.

In an alternative embodiment, the heart valve flaps can be worked onto a woven or knitted vascular prosthesis or one produced by the melt-blown technique, e.g. of PET or PVDF (polyvinylidene fluoride). In particular, a vascular prosthesis according to the prior art can be used as the tubular portion.

The implant, in particular the tubular portion of the heart valve prosthesis, can advantageously be provided with a reinforcement, in particular in the form of a textile construction. The reinforcement can preferably be in the form of a braid. In another embodiment, the reinforcement can be designed as a fiberwoven fabric radially and around the circumference of the flaps. Advantageously, the reinforcement can be made of monofilaments arranged in the principal direction of deformation. Moreover, the reinforcement can be made on the basis of polyethylene terephthalate (PET).

Particularly advantageously, the implant according to the invention is designed for colonization with living cells, in particular human cells. Such cell colonization can be in one layer or several layers. In this way, it can serve as a flexible framework for production of a biohybrid implant colonized with cells. The nonwoven-type surface structure and the choice of material for formation of a cardiovascular implant permit ready adherence of cells to the surfaces of the implant. A cell layer with good adherence properties is able to form. If appropriate, several cell layers can preferably be superposed on one another, and in particular the cell layers can differ from one another in terms of different cell types. Growth of cells into the implant can be favored or impeded by appropriate choice of the microporous structure. By provision of framework or blank for a heart valve prosthesis, it is possible to produce in situ and in a simple manner a biohybrid implant colonized with cells from the patient concerned.

In a particular embodiment of the invention, the implant can be designed as a biohybrid cardiovascular implant. The implant according to the invention can be characterized by the fact that it is colonized with human cells at least partially, in particular in several layers. Colonizing cells can be anchored on the nonwoven. In particular, the colonizing cells can be formed as a flat cell lawn. The cells can preferably be layered. In a preferred embodiment of the implant according to the invention, colonizing cells can cover the surface, directed toward the blood, as a flat cell sheet.

Advantageously, the colonizing cells can be chosen from the group of fibroblasts, smooth muscle cells and endothelial cells. In a further development, colonizing cells can also be chosen from the group of differentiated stem cells and cloned cells. According to the invention, cells of the implant which come into contact with the patient's blood stream can be endothelial cells. In particular, the endothelial cells can colonize a cell layer containing fibroblasts and/or smooth muscle cells. An intact endothelium influences the blood clotting behavior so that even clotting parameters which have already been activated are now once more deactivated. The cell layer lying under the endothelial cells in the implant according to the invention can act as an adherence layer. The adherence of the endothelium to the substrate is of crucial importance for its functioning. If the adherence is inadequate, there is a danger that the cells will be entrained with the blood stream and, in this way, a foreign implant surface will be exposed. The colonizing cell sheet can advantageously have a high shear strength, that is to say the colonizing cell sheet is able to remain stable under the physiologically occurring shear forces.

The implant according to the invention can be distinguished by the fact that the colonizing cell sheet deactivates clotting parameters in the blood. The action of clotting substances possibly formed as a reaction to the implantation can thus be inhibited. For the patient, therefore, the risks associated with clotting and thickening (coagulation) of the blood and with thrombus formation following a cardiovascular intervention are greatly reduced. The use of anticlotting and blood-thinning medicines (anticoagulents) can remain restricted to a minimum or can be dispensed with altogether.

The colonizing cells can preferably be cultivated from cells originating from the implant recipient. In this way it is possible to minimize foreign-body reactions in the patient being treated. The implant made from a preform of synthetic polymer material is concealed under a cell colony of endogenous cells from the patient, with the result that the body's immune response recognizes the implant as “own” material, not as “foreign” material, and thus suppresses defense reactions. For the patient, this affords the advantage of a long-lasting function of the implant. At the same time, the administration of medicines for suppressing foreign-body reactions and defense reactions can be kept to a minimum or dispensed with altogether.

The present invention also relates to a method for producing a biohybrid implant, made substantially of biocompatible synthetic polymer material in the form of a three-dimensional structure, wherein a preform of the implant made of synthetic polymer material is colonized with human cells, initially with fibroblasts and/or smooth muscle cells, then, preferably after a rest phase for anchoring of the first cell layer, with colonization of endothelial cells in a surface cell layer.

Preferably, endothelial cells, fibroblasts and smooth muscle cells can be obtained from explanted sections of the great saphenous vein of the patient to be treated and are cultivated in specific cell media using human serum from the patient. The technique of collecting cells and cultivating them is familiar to the skilled persons, so that no description is needed here.

According to the invention, a cardiovascular implant can be colonized with human cells both on the inside and on the outside. In particular, the inside can be completely lined with endothelial cells. Moreover, the outside of the implant, in particular of the heart valve prostheses, can be colonized with smooth muscle cells and fibroblasts. Through the cell layer, the wall of the implant in the area particularly exposed to mechanical loads is additionally strengthened and is protected against inflammatory reactions of the patient's body. The flaps preferably bear only a thin layer of fibroblasts with a confluent layer of endothelial cells.

In tests using the implant according to the invention, it was found that preliminary colonization with fibroblasts or a mixed culture of fibroblasts and smooth muscle cells, even with a slight loss of endothelial cells, activates blood coagulation much less than is the case where a surface lies exposed as a foreign body. This can be attributed to the fact that small defects in the endothelial cell layer are compensated, or repaired as it were, on such a physiological substrate.

If so required, the cells can be obtained from precursor cells or stem cells.

During and/or after its production, the heart valve can advantageously be connected to tubular connection pieces which are used for connection to the heart and to the aortic arch.

The invention also makes available a device for producing a biohybrid implant, in particular for carrying out the method according to the invention, this device being characterized by an incubator which is designed for cell colonization of a preform of a surgical implant made of synthetic polymer material and which receives the cell medium, in particular a cell suspension, rotatable about at least one axis, preferably about several axes, and a support which can be placed in it for receiving and fixing the implant to be coated with cells.

So that the cells are able to colonize homogeneously on the corresponding implant, the device in question is a round vessel which can be rotated about its own axis. The implant to be coated is fixed in an inlay designed as a holding frame. In one embodiment, the incubator can for example receive 50 to 200 ml of cell medium and 5 to 20 ml of cell suspension with 10⁶ to 10⁸ cells. With the aid of a rotation machine, the incubator can be rotated for several hours. The speeds of rotation can be between less than 1 and 30 revolutions per minute, preference being for 1 to 10 revolutions per minute. Rotation phases can advantageously alternate with stationary phases of about 10 to 60 minutes, in particular 15 to 45 minutes. The stationary phases allow the cells to settle by gravity on the substrate, or implant surface. During the rotation phases, cells which have not yet adhered are brought back into suspension and distributed in the incubator. The speed of rotation is chosen such that, after each rotation, another part of the implant surface is oriented upward and thus exposed for cell colonization. This is achieved by the fact that the respectively last revolution of a rotation cycle is not executed completely, but only to the extent of 351 to 359°.

In another embodiment of the device according to the invention, the incubator can additionally turn clockwise. By this means, the inlay moves upon each 180° turn, so that, after each 360° rotation, there is a small flow of medium with cell suspension through the three-dimensional implant to be colonized.

Rotation phases and stationary phases can be chosen according to cell type, cell density and desired cell layer thickness, so as to be able to achieve a desired distribution of the cells on the substrate.

To check the resistance of the cell layers to shear forces, an in vitro test is carried out in a perfusion chamber, by which means it is possible to conduct standard tests of a number of states which arise in the body of a patient. The heart valve prosthesis to be tested is lengthened with two vascular prostheses and fixed in the perfusion chamber so that it lies free in the test medium, cell medium or bloodgroup-compatible blood. A pulsatile pump is used to convey the perfusate in the physiological direction through the prosthesis, it being possible to set a frequency of between 60 and 120 pulses per minute. A second pump is used to continuously suction a volume of about 100 ml/min from the proximal end of the valve, so as to bring about valve closure. The movements of the valve flaps impose a particular load on the cell coating. The valves are tested at pressures values of between 100 and 200 systolic and at 30 to 80 diastolic. During the test, the action of the valve flaps can be recorded by a camera introduced in front of the valve.

The present invention also concerns the provision of a surgical implant for use as vascular prosthesis in cardiovascular surgery. Narrow-lumen bypass vessels with diameters in the range of up to 5 millimeters obtained from the implant according to the invention can particularly advantageously be used. In known prostheses for aortocoronary bypass with small vessel diameter, the foreign surface has a thrombogenic action, leading to early activation of the coagulation cascade and to thrombotic occlusion of the vessel. The inner endothelial surface formed according to the invention by means of the patient's cells is able to prevent thrombus form ation and, consequently, occlusion of the implanted vessels.

For a further application, the implant according to the invention can be provided as a heart valve prosthesis in cardiac surgery. The heart valve prosthesis according to the invention can particularly advantageously be used as an implant in the high-pressure circulation of the left portion of the heart. Implantation of heart valve prostheses colonized inside and outside with autologous cells is of advantage to the patient in several respects. Since the inner surface which comes into contact with the blood stream is composed of the patient's own endothelial cells and thus avoids formation of blood clots, anticlotting medication is superfluous. In addition, no immunological reactions such as rejection are to be expected, because only the patient's own cells come into contact with the immune system of the patient.

Further features and details of the invention will become evident from the following description of preferred illustrative embodiments. The individual features can in each case be realized singly, or severally in combination. The examples serve only to explain the present invention and are not intended to limit it in any way.

### EXAMPLES

### Example 1

Use of the Colonizing Device for Cell Colonization of an Implant

The incubator, with the implant to be coated fixed in it, is charged with 100 ml of cell medium and 10 ml of cell suspension, containing 10⁶ to 10⁸ cells, and held for 12 to 24 hours in a rotation machine. In this machine, during the coating operation, stationary phases of 15 to 45 minutes alternate with rotation phases of 1 to 10 minutes. The speeds of rotation are between 1 and 10 revolutions per minute. The duration of the stationary phases and movement phases and the speeds of rotation can be varied depending on the cell type used, the cell density, and the desired cell layer thickness. The stationary phases allow the cells to settle by gravity on the substrate. During the rotation phases, cells which have not yet adhered are brought back into suspension and distributed in the incubator. The speed of rotation can be set such that after each rotation another part of the implant surface is oriented upward and thus exposed for cell colonization. This is achieved by the fact that the respectively last revolution of a rotation cycle is not executed completely, but only to the extent of 331 to 359°.

### Example 2

Use of the Colonizing Device for Cell Colonization of Heart Valve Flaps

In a heart valve prosthesis, the valve flaps are much thinner than the corresponding wall of the aorta, so that a cell layer colonizing on the valve flaps must also be thinner. The valve flaps ought also to be colonized with cells on both sides. In the incubator, configured as in Example 1 above, the preform of the heart valve prosthesis to be colonized is fixed in the inlay. The filling volume is 100 ml, including 10 ml of cell suspension containing 10⁶ to 10⁷ cells. The incubation time is 12 to 24 hours, the rotation phases 1 to 10 minutes, and the rest phases 15 to 45 minutes. During the incubation, the device is additionally turned clockwise, by which means the inlay moves on each turn through 180°, and upon each rotation through 360° there is a small flow through the valve flaps, causing corresponding movements of these. The effect of this movement is that there is an exchange of cell suspension above and below the flaps, by which means the cell colonization is more homogeneous. Here, in the same way as in Example 1, the rotations of the incubator always end in another position, so that, in each rest phase, another part of the surface is exposed.

### Example 3

Cell Colonization of a Vascular Prosthesis—Variant 1

Using the colonizing device described above in Example 1, an implant designed as a vascular prosthesis is colonized first with fibroblasts. This is followed by a rest period of 2 to 8 days in culture medium, in order to ensure that the cells have grown onto the surface and adhere to it. This is dependent on the initial coating density and on the adhesion on the prosthesis. After this phase, colonization with endothelial cells is performed. After a further rest phase of 2 to 7 days, the prosthesis is ready to be implanted in the patient.

### Example 4

Cell Colonization of a Vascular Prosthesis—Variant 2

Using the colonizing device described above in Example 1, an implant designed as a vascular prosthesis is colonized first with a mixed culture of smooth muscle cells and fibroblasts. This is followed by a rest period of 2 to 8 days in culture medium, in order to ensure that the cells have grown onto the surface and adhere to it. This is dependent on the initial coating density and on the adhesion on the prosthesis. After this phase, colonization with endothelial cells is performed. After a further rest phase of 2 to 7 days, the prosthesis is ready to be implanted in the patient.

### Example 5

Cell Colonization of an Aortic Valve—Variant 1

In a manner analogous to the method of Example 3, the outer wall and inner wall of a preform of a prosthetic aortic tube is coated with fibroblasts (cell suspension with >10⁷ cells) by means of the incubator from Example 1. After a rest phase of one day, renewed coating with fibroblasts is carried out using the device from Example 2 and with a smaller cell count of 10⁵ to 10⁶ cells in order to generate a corresponding cell layer also on the valve flaps. This is followed by a rest phase of 2 to 8 days. Thereafter, the aortic prosthesis is colonized with endothelial cells in the incubator from Example 2. After a further rest phase of 2 to 7 days, the prosthesis is then ready to be implanted in the patient.

### Example 6

Colonization of an Aortic Valve—Variant 2

In a manner analogous to the method of Example 3, the outer wall and inner wall of a preform of a prosthetic aortic tube is coated with a mixed culture of fibroblasts and smooth muscle cells (>10⁷ cells) in the incubator from Example 1. After a rest phase of one day, renewed coating with fibroblasts is carried out using the incubator from Example 2 and with a smaller cell count (10⁵ to 10⁶ cells) in order to generate a corresponding cell layer also on the flaps. This is followed by a rest phase of 2 to 8 days, then colonization with endothelial cells in the incubator from Example 2. After a further rest phase of 2 to 7 days, the prosthesis is then ready to be implanted in the patient.

### Example 7

Embodiment of a Heart Valve Prosthesis

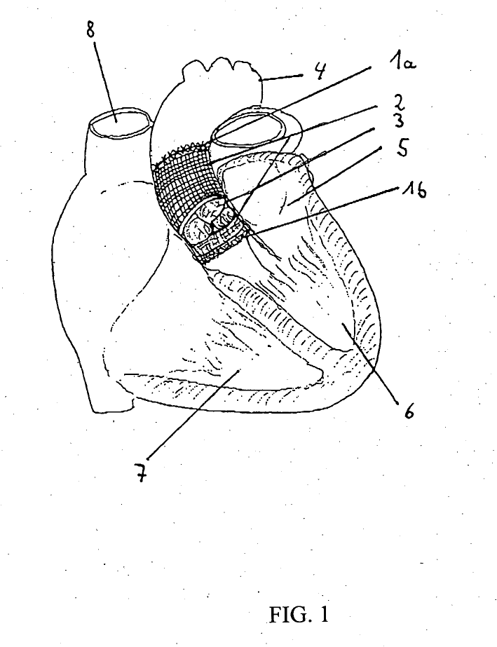

To explain the illustrative embodiment, reference is made to attached FIG. 1 which shows a heart valve prosthesis which, for the sake of clarity, is partially opened. The symbols in the drawing have the following meanings: 1a/b suture rings; 2 aortic stump prosthesis; 3 arterial valve prosthesis; 4 aorta; 5 left atrium; 6 left ventricle; 7 right ventricle; 8 superior vena cava.

The implant is formed with flaps made of polyurethane nonwoven and with a woven/knitted vascular prosthesis made of polyester. A woven aortic stump prosthesis (2) was fixed on a core which holds the woven tube open and constitutes a model of the inner contours of a heart valve with aortic stump and closed flaps. By spraying a polyurethane solution onto the tube and onto the core in the tube, the heart valve prosthesis (3) can be integrated directly into the aortic stump prosthesis. The fine (delicate) nonwoven structure, which forms the heart valve prosthesis and connects it to the aortic stump, can thus be made to pentrate into the woven aortic stump prosthesis. The suture rings 1a and 1b are formed from a nonwoven tube which has been sprayed from polyurethane solution and then rolled up. After removal of the core, the flaps are separated from one another by incisions. They remain in the closed postion in the unloaded state.

1-26. (canceled)

27. A cardiovascular implant for use in surgery made substantially of biocompatible synthetic polymer material in the form of a three-dimensional structure, wherein the polymer material is substantially nonabsorbable under physiological conditions, and the implant, at least on part of its surface, is formed as a microporous nonwoven from microfibers of the polymer material, which permit colonization with cells.

28. The implant as claimed in claim 27, wherein the implant is a heart valve with flaps.

29. The implant as claimed in claim 27, wherein the nonwoven has a thickness of 10 to 500 μm.

30. The implant as claimed in claim 29, wherein the nonwoven has a thickness of 30 to 70 μm.

31. The implant as claimed in claim 27, wherein the microfibers have a thickness of 0.1 to 10 μm.

32. The implant as claimed in claim 27, wherein the nonwoven has a pore size in the range of 0.1 to 20 μm.

33. The implant as claimed in claim 32, wherein the pore size is in the range of 1 to 8 μm.

34. The implant as claimed in claim 28, in the form of a heart valve prosthesis comprising a tubular portion as a stump of a cardiac blood vessel which is designed to be sutured onto an end piece of a natural blood vessel, three flaps which form a heart valve being arranged movably relative to one another on the stump and interacting with one another as a valve so as to open and close upon flow of blood.

35. The implant as claimed in claim 34, wherein the cardiac blood vessel is an aortic stump.

36. The implant as claimed in claim 28, wherein the heart valve flaps are made from a planar nonwoven.

37. The implant as claimed in claim 28, wherein the heart valve flaps are arched.

38. The implant as claimed in claim 34, wherein the implant has a wall thickness of 1 to 3 mm in the area of the vessel portion.

39. The implant as claimed in claim 28, wherein the heart valve flaps are produced as separate parts and are secured on the tubular portion of the prosthesis.

40. The implant as claimed in claim 39, wherein the parts are secured by one or more of adhesively bonded and melted.

41. The implant as claimed in claim 28, wherein the heart valve prosthesis is made in one piece with the flaps.

42. The implant as claimed in claim 41, wherein the tubular portion of the heart valve prosthesis is made in one piece with the flaps.

43. The implant as claimed in claim 34, wherein the heart valve flaps and the tubular portion have a different structure.

44. The implant as claimed in claim 34, wherein the heart valve flaps and the tubular portion are made from different materials.

45. The implant as claimed in claim 34, wherein the heart valve prosthesis is provided with a reinforcement.

46. The implant as claimed in claim 45, wherein the reinforcement is provided in the tubular portion of the heart valve prosthesis.

47. The implant as claimed in claim 49, wherein the reinforcement is in the form of a textile construction.

48. The implant as claimed in claim 27, wherein the implant is produced at least partially by a spray-bonding technique.

49. The implant as claimed in claim 27, wherein the implant is desgined for colonization with living cells.

50. The implant as claimed in claim 49, wherein the cells are human cells.

51. The implant as claimed in claim 50, wherein the implant is colonized with human cells at least partially.

52. The implant as claimed in claim 49, wherein the implant is colonized with living cells in several layers.

53. The implant as claimed in claim 49, wherein colonizing cells cover the surface, directed toward the blood, as a flat cell sheet.

54. The implant as claimed in claim 49, wherein colonizing cells are chosen from the group of fibroblasts, smooth muscle cells and endothelial cells.

55. The implant as claimed in claim 54, wherein the endothelial cells are colonized on the implant on a cell layer containing one or more of fibroblasts and smooth muscle cells.

56. The implant as claimed in claim 53, wherein the colonizing cell sheet has a high shear strength.

57. A method for producing a biohybrid implant made substantially of biocompatible synthetic polymer material in the form of a three-dimensional structure with a nonwoven-type surface, wherein a preform of the implant made of synthetic polymer material is colonized with human cells, initially with one or more of fibroblasts and smooth muscle cells, then colonized with endothelial cells in a surface cell layer.

58. The method as claimed in claim 57, wherein colonization of endothelial cells is performed after a rest phase for anchoring of the first cell layer.

59. The method as claimed in claim 57, wherein endothelial cells, fibroblasts and smooth muscle cells are obtained from explanted sections of the great saphenous vein of the patient to be treated and are cultivated in specific cell media using human serum from the patient.

60. The method as claimed in claim 57, wherein the heart valve, during and/or after its production, is connected to tubular connection pieces which are used for connection to the heart and to the aortic arch.

61. A device for producing a biohybrid implant, carrying out the method as claimed in claim 57, comprising an incubator which is designed for cell colonization of a preform of a surgical implant made of synthetic polymer material and which receives the cell medium, rotatable about at least one axis, and a support which can be placed in it for receiving and fixing the implant to be coated with cells.

62. Provision of the implant as claimed in claim 27 as a vascular prosthesis in cardiovascular surgery.

63. Provision of the implant as claimed in claim 27 as a heart valve prosthesis in cardiac surgery.
